(19) United States
(12) Patent Application Publication
(10) Pub. No.: US 2026/0083132 A1
(43) Pub. Date: Mar. 26, 2026

(54) THIOL-CONTAINING ALCOHOLS FOR GENERATING NITRIC OXIDE AND METHODS OF FORMING THE SAME

(71) Applicant: Sterile State, LLC, Farmington Hills, MI (US)

(72) Inventor: Megan Cecelia Frost, Hancock, MI (US)

(73) Assignee: Sterile State, LLC, Farmington Hills, MI (US)

(21) Appl. No.: 19/126,048

(22) PCT Filed: Nov. 8, 2023
(86) PCT No.: PCT/US2023/079150

§ 371 Date: Apr. 30, 2025

Related U.S. Application Data
(60) Provisional application No. 63/428,048, filed on Nov. 26, 2022.

(51) Int. Cl. A01N 59/00; A01N 25/10; A01P 1/00; A61L 2/20; A61L 101/02; C07F 7/21

(52) U.S. Cl. CPC A01N 59/00; A01N 25/10; A01P 1/00; A61L 2/20; C07F 7/21; A61L 2101/02

(57) ABSTRACT

Compositions and methods are presented for a nitric oxide precursor that decomposes to thereby release nitric oxide. The nitric oxide precursor includes a reaction product of a thiol-containing alcohol, a silane, and a nitrosating compound that allows for technically simple (e.g., solventless 1-pot synthesis) and flexible synthesis of nitric oxide precursors and mixtures thereof that allow for controlled release rate of nitric oxide (e.g., via use of primary, secondary, and/or tertiary nitrosothiols, alone or in combination with additives).

### CROSS REFERENCE TO RELATED APPLICATIONS

This application claims priority to our US provisional patent application with the Ser. No. 63/428,048, which was filed Nov. 26, 2022, and is incorporated by reference herein.

### FIELD OF THE INVENTION

The present disclosure generally relates to a nitric oxide precursor comprising a reaction product of a thiol-containing alcohol, a silane, and a nitrosating compound.

### BACKGROUND OF THE INVENTION

A variety of products and articles, including, for example, medical instruments, devices, and equipment, must be sterilized prior to use to prevent bio-contamination of a wound site, a sample, an organism, or the like. A number of sterilization processes are used which involve contacting the product or article with a sterilant. Examples of such sterilants include dinitrogen tetroxide, nitric oxide, steam, ethylene oxide, hydrogen peroxide, dry heat, and the like. For example, where the sterilant is nitric oxide, most conventional methods use catalytic or enzymatic generation of nitric oxide (NO) from nitrite or a NO donating compound such as diazeniumdiolates, typically requiring a relatively complex system for generation, maintenance, and disposal of the nitric oxide.

Alternatively, nitric oxide can be produced at the site of use from a precursor compound that releases upon decomposition the nitric oxide. Most typically, such precursors include a chemically labile nitrosothiol group. For example, in WO 2017/156078, an implantable device comprising nanostructured lipid particles capable of releasing nitric oxide is presented. Here, the inventors used the thiol-lipid DPPTE (1,2-dipalmitoyl-sn-glycero-3-phosphothioethanol) that after nitrosylation was incorporated lipid nanoparticles for treatment of a subject having a nitric oxide-mediated disorder. Although conceptually interesting, such nitric oxide precursors have been limited to in vivo use with lipid-based therapeutics. In addition, and aside from the complex manner of manufacture, the concentration of nitric oxide from such precursors is generally insufficient for sterilization in an ex vivo situation.

In other known approaches, selected polymers were reported that can be modified to provide nitrosothiol groups in amounts that are useful for NO based sterilization processes. For example, in WO 2023/205125, modified polymeric materials were prepared to include pendant nitrosothiol groups from which nitric oxide can be released. Here, polycarbonate-polydimethylsiloxane (PCPDMS) block co-polymer, polyurethane-polydimethylsiloxane (PUPDMS) block co-polymer, polyurethane (PU), poly(ethylene-co-vinyl acetate) copolymer (EVA), or polydimethylsiloxane were first reacted with 3-aminopropyl trimethoxy siloxane to form a pendant amine group that is then reacted with acetylpenicillamine thiolactone to introduce a thiol group that is subsequently reacted with t-butyl nitrite to convert the exposed thiol group to the corresponding S-nitrosothiol. Unfortunately, such an approach is limited to specific reactive groups that are only present in the polymer.

Moreover, these known methods for forming nitric oxide precursors will require expensive reactants and multiple reaction sequences, rendering synthesis relatively complex. Furthermore, small molecule nitrosothiols that decompose to form nitric oxide are endemically unstable, especially when in solution, and typically provide no flexibility in making primary, secondary, or tertiary RSNOs. Still further, due to the chemical nature of the currently known precursors, composite materials containing these precursors are often limited.

Accordingly, there remains a need for improved compositions and methods that allow for simple and flexible synthesis of nitric oxide precursors, especially where such precursors can be used in a wide variety of uses, shapes, and forms, and/or where such precursors allow tuning of the rate of release nitric oxide.

### SUMMARY OF THE INVENTION

The inventive subject matter is directed to various compositions and methods for nitric oxide precursors that allow for technically simple (e.g., solventless 1-pot synthesis) and flexible synthesis of nitric oxide precursors and mixtures thereof that allow for controlled release rate of nitric oxide (e.g., via use of primary, secondary, and/or tertiary nitrosothiols, alone or in combination with additives). Advantageously, the nitric oxide precursors presented herein can be made in numerous forms, shapes, and sizes, can be easily compounded with/into other materials.

In one aspect of the inventive subject matter, the inventor contemplates a composition comprising a carrier that includes a polymeric material, or an inorganic material coupled to or in admixture with a compound having a structure according to Formula I:

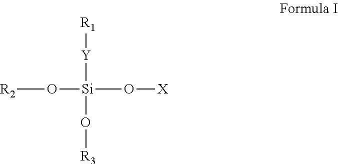

Preferably, but not necessarily, X is selected from the group consisting of:

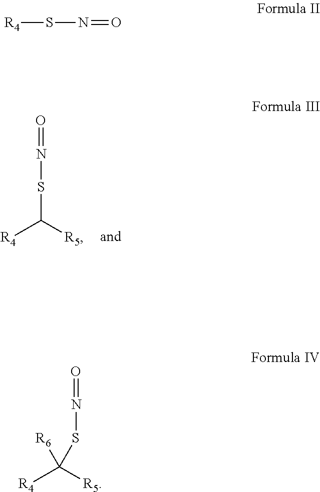

wherein X is covalently bound to O in Formula I via the R4 group in Formula II-IV. Most typically, Y is O or null, each of R1, R2, R3, R4, R5, and R6 are independently selected from the group consisting of hydrogen, alkyl, alkoxy, alkenyl, alkynyl, cycloalkyl, hetercycloalkyl, and aryl, and optionally each of R1, R2, R3, R4, R5, and R6 are, when not a hydrogen, independently substituted with a thiol group, an epoxy group, a nitro group, and/or an amino group.

In some embodiments, the carrier is a polymeric carrier that at least partially coats the compound. For example, a suitable carrier includes a synthetic polymeric carrier that is configured as a packaging material, a film, or a pellet. In additional example, a suitable carrier also includes a natural polymeric carrier that is configured as a fiber, a cloth, or a woven article.

In further embodiments, the compound is granulated and has a largest dimension of no more than 500 μm. Preferably, but not necessarily, the compound is present in the composition in an amount of between 0.01 wt % and 0.1 wt %. Nonetheless, the compound is typically present in the carrier in an amount sufficient to release antimicrobially effective quantities of nitric oxide from the carrier. It is further generally contemplated that the compound is covalently coupled to the carrier.

In further contemplated embodiments, X has a structure according to Formula II, or X has a structure according to Formula III, or X has a structure according to Formula IV. Alternatively, or additionally, the composition comprises a compound of Formula I in which Y is null, and further comprises an additional compound of Formula I in which Y is O. For example, the composition includes a compound of Formula I in which X has a structure according to one of Formula II, III, or IV, and further includes an additional compound of Formula I in which X has a structure according to one of Formula II, III, or IV, wherein Y in the compound of Formula I is null, and wherein Y in the additional compound is O. Moreover, X in the compound and X in the additional compound may not be same.

In some embodiments, the composition further comprises an additive. In such case, the additive is selected from the group consisting of a pH modulator, a hygroscopic agent, a hydrophobic agent, a light filter, a light sensitizer, a transition metal, a chelator, glutathione, and a reducing agent.

Viewed from a different perspective, the inventor additionally contemplates a method of forming a nitrosothiol-containing nitric oxide precursor that includes combining in a reaction vessel a thiol-containing alcohol, a silane, and a nitrosating compound to form a reaction mixture, and reacting in the reaction mixture the thiol-containing alcohol with the silane to thereby form a thiol-containing intermediate and reacting in the reaction mixture the thiol-containing intermediate with the nitrosating compound to thereby form the nitrosothiol-containing nitric oxide precursor.

As desired, the thiol-containing alcohol comprises a primary thiol, or the thiol-containing alcohol comprises a secondary thiol, or the thiol-containing alcohol comprises a tertiary thiol.

In various embodiments, the thiol-containing alcohol is selected form the group consisting of 3-mercapto-3-methyl-1-butanol, 2-mercaptoethanol, 3-mercaptohexanol, 4-mercapto-3-methyl-2-butanol, 3-mercapto-2-methyl-1-butanol, 3-mercapto-2-methyl-1-pentanol, (+/−)-4-mercapto-4-methyl-2-pentanol, 2-mercapto-2-methyl-1-pentanol, 4-mercapto-4-methylpentan-2-ol, 3-mercaptohexan-1-ol, mercaptoethanol, 1-mercapto-3-propanol, 1-mercapto-4-butanol, and an α-mercapto-ω-hydroxyoligoethylene oxide.

In further embodiments, the silane is a tetraalkoxysilane, a trialkoxysilane, or a cyclic azasilane. Alternatively, or additionally, the silane is selected from the group consisting of tetraethylorthosilicate (TEOS), methyltrimethoxysilane (MTMS), vinyl-trimethoxysilane, methylvinyldimethoxysilane, dimethyldiethoxysilane, vinyltriethoxysilane, tetra-n-propyl-orthosilicate, trisacetamidomethylsilane, bisacetamidodimethylsilane, methylmethoxybis-(ethylmethylketoximo)silane, methyldimethoxy-ethylaminosilane; dimethyldi-N,N-di-methylaminosilane, methyldimethoxyisopropylaminosilane triacetoxyvinylsilane, tris-(2-methoxyethoxy)-vinylsilane, 3-chloropropyltriethoxysilane, 3-mercaptotriethoxysilane, ethyltrimethoxysilane, phenyltriacetoxysilane, methyltrimethoxysilane, and phenyltri-methoxysilane.

In some embodiments, the nitrosating compound is an inorganic nitrite salt. In further embodiments, the nitrosating compound is selected from the group consisting of sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, an ion paired nitrite, silver nitrite, zinc nitrite, iron nitrite, copper nitrite, and a transition metal-nitrite compound.

Advantageously, the steps of reacting the thiol-containing alcohol with the silane and reacting the thiol-containing intermediate with the nitrosating compound does not require a solvent to form the nitrosothiol-containing nitric oxide precursor. Moreover, the steps of reacting the thiol-containing alcohol with the silane and reacting the thiol-containing intermediate with the nitrosating compound can be performed without adjustment of pH. Alternatively, or additionally, the steps of reacting the thiol-containing alcohol with the silane and reacting the thiol-containing intermediate with the nitrosating compound are performed at ambient temperature and ambient pressure. Some embodiments include an additional step of removing the nitrosothiol-containing nitric oxide precursor by filtration. Further embodiments include adding at least one of an acid and a catalyst to the reaction mixture, wherein the acid is acetic acid and wherein the catalyst is di-n-butyldilauryltin. Preferably, but not necessarily, the nitrosothiol-containing nitric oxide precursor has a structure according to Formula I.

Viewed from an additional perspective, the inventor further contemplates a method of sterilization that comprises placing a non-sterile object in a confined space, wherein the confined space further includes the contemplated NO-releasing composition, sealing the confined space, wherein the non-sterile object and the contemplated NO-releasing composition are physically separated from an environment external to the confined space, and releasing nitric oxide (NO) into the confined space from the contemplated NO-releasing composition.

As desired, the non-sterile object may comprise a medical device, a bandage, a dressing, an electronic device, a surgical equipment, a tissue sample, a biohazardous material, a recreational equipment, a kitchen product, and/or a cleaning tool.

Preferably, but not necessarily, the confined space is configured as a bag, a pouch, or a container with a lid, or wherein the entire confined space is flexible. In further embodiments, the confined space has a volume between 10 cm³ and 1,000 cm³. Most typically, the confined space is configured to allow sealing using heat, ultrasonic energy, laser irradiation, an adhesive, or manual sealing. Moreover, the contemplated NO-releasing composition releases NO in response to a change in temperature, pressure, pH, humidity, illumination with visible or UV light, or a combination thereof.

In additional embodiments, the contemplated NO-releasing composition is present in an amount sufficient to sterilize via gaseous NO the non-sterile object contained in the confined space. Most typically, NO is released for an amount of time sufficient to sterilize the non-sterile object in the confined space at room temperature or at elevated temperature. For example, sterilization is performed to a sterility assurance level of equal or less than 10⁻⁶.

Therefore, the inventor also contemplates a method of forming a composite article configured to sterilize objects that includes forming or obtaining the contemplated composition, incorporating the composition into an article that is configured to retain a non-sterile object, thereby forming the composite article, wherein the composite article is configured to allow for delivery of nitric oxide to the article in an amount sufficient for sterilization.

As will be appreciated, the step of forming may comprise pressing or extruding the composition or combining the composition with a packing material. In some embodiments, the packing material is a thermoplastic polymeric material. Preferably, but not necessarily, the composite article is configured as a bag, a tray, or a flask.

As desired, the step of incorporating comprises enclosing the composition into a gas permeable sachet and placing the sachet into the article. In further embodiments, incorporating comprises combining the composition with a packing material and forming the article from the combination. As noted above, sterilization is preferably performed to a sterility assurance level of equal or less than 10⁻⁶.

Various objects, features, aspects, and advantages of the disclosure will become more apparent from the following detailed description of preferred embodiments, along with the accompanying drawing figures in which like numerals represent like components.

### BRIEF DESCRIPTION OF THE DRAWING

FIG. 1 is an image illustrating non-limiting embodiments of reaction schematics for forming a nitric oxide precursor.

FIG. 2 is an image illustrating non-limiting embodiments of chemical structures of a nitric oxide precursor.

FIG. 3 are images and graphs illustrating non-limiting embodiments of a nitric oxide precursor and a composite article including the same.

FIG. 4 are images and graphs illustrating non-limiting embodiments of a nitric oxide precursor and a composite article including the same.

FIG. 5 are images and graphs illustrating non-limiting embodiments of a nitric oxide precursor and a composite article including the same.

FIG. 6 are images and graphs illustrating non-limiting embodiments of a nitric oxide precursor and a composite article including the same.

### DETAILED DESCRIPTION

The inventor has provided a reaction system that uses a solventless, 1-pot synthesis, to react thiol-containing alcohol compounds with silanes, with or without a metallo-catalyst such as dibutyltin dilaurate or dimethyldineodecanoate tin and a nitrosating agent such as tert-butylnitrite, to allow for flexible synthesis of nitric oxide precursors and mixtures thereof that control release rate of nitric oxide.

In various embodiments the reaction system will produce a nitric oxide precursor to which a carrier (e.g., comprising a polymeric material or an inorganic material) can be covalently coupled to, or (homogeneously or heterogeneously) mixed with. Preferably, the nitric oxide precursor will have a structure according to Formula I as shown below.

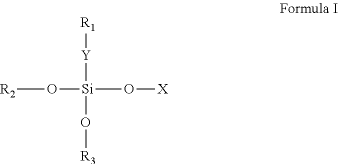

In some embodiments, the nitrosothiol-containing nitric oxide precursor has a structure according to Formula I.

As can be readily appreciated, X is most typically selected from the group consisting of Formula II, Formula III, and Formula IV, wherein X is covalently bound to O in Formula I via the R4 group in Formula II-IV.

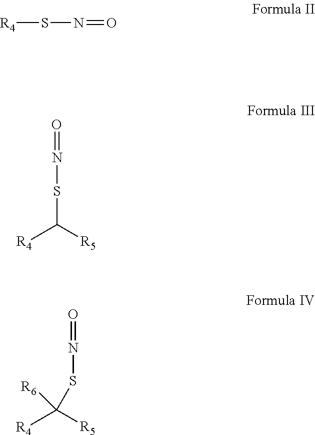

As desired, Y is O or null. In addition, each of R1, R2, R3, R4, R5, and R6 are independently selected from the group consisting of hydrogen, alkyl, alkoxy, alkenyl, alkynyl, cycloalkyl, hetercycloalkyl, and aryl. Preferably, but not necessarily, where each of R1, R2, R3, R4, R5, and R6 are, when not a hydrogen, independently substituted with a thiol group, an epoxy group, a nitro group, and/or an amino group.

In some embodiments, the contemplated composition comprises a compound of Formula I in which X has a structure according to one of Formula II, III, or IV, and further comprises an additional compound of Formula I in which X has a structure according to one of Formula II, III, or IV, wherein X in the compound and X in the additional compound are not the same.

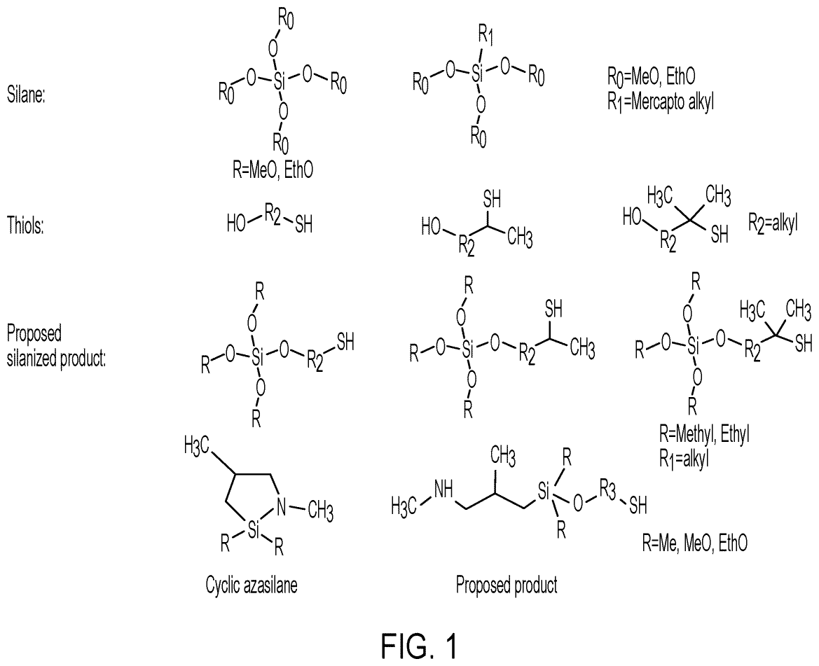

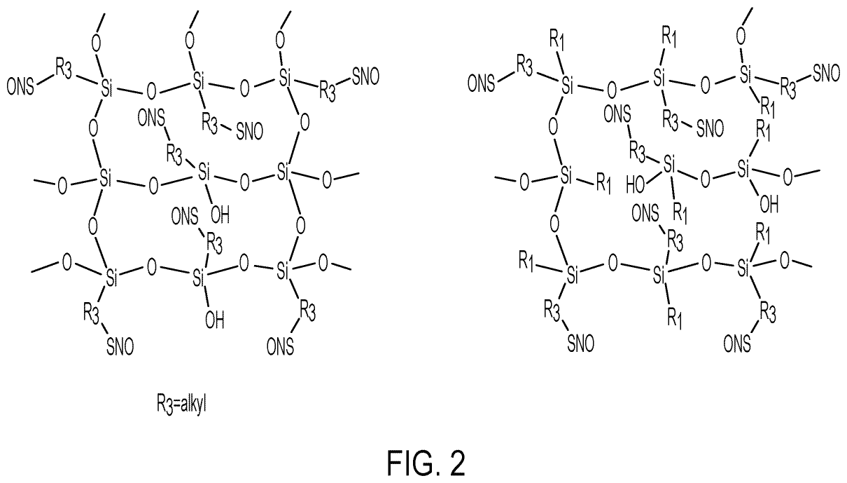

Most typically, the (primary, secondary, tertiary) thiol-containing alcohol and the silane can react to form an intermediate, and the intermediate and the nitrosating compound are capable of reacting to form the nitric oxide precursor. Exemplary reaction schematics (A), (B), and (C) are shown below and in FIG. 1. Exemplary nitric oxide precursors are shown in FIG. 2.

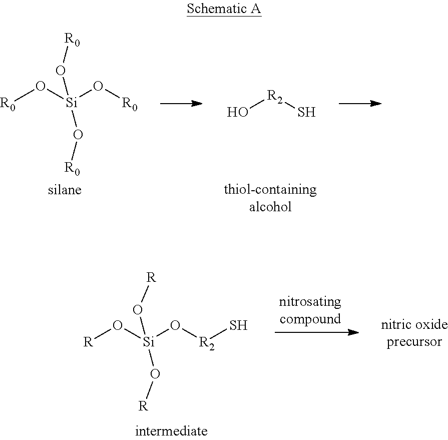

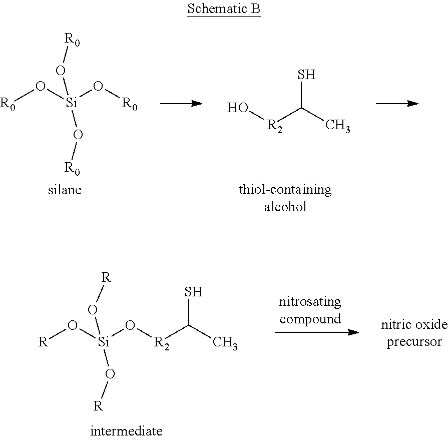

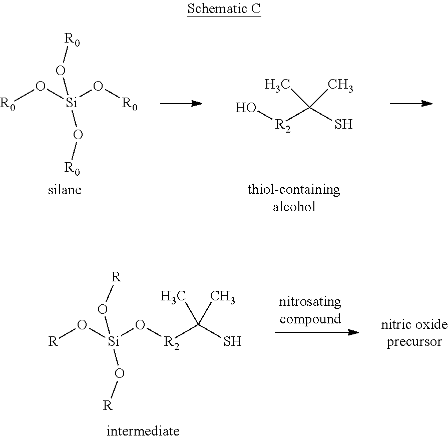

Preferably, but not necessarily, R is, independently, a methyl group, a methylene oxide group, or an ethylene oxide group, each R0 is, independently, a methylene oxide group or an ethylene oxide group, R1 is a mercapto alkyl group, and each R₂ is, independently, an alkyl group.

As desired, the components utilized to form the reaction product may be combined in a reaction mixture. It is to be appreciated that the components of the reaction mixture are not yet reacted with each other. The reaction mixture for forming the reaction product may comprise the thiol-containing alcohol in an amount of from about 1 to about 80 wt. %, alternatively from about 1 to about 70 wt. %, or alternatively from about 50 to about 80 wt. %, based on a total weight of the reaction mixture. The reaction mixture for forming the reaction product may comprise the silane in an amount of from about 1 to about 80 wt. %, alternatively from about 1 to about 70 wt. %, or alternatively from about 50 to about 80 wt. %, based on a total weight of the reaction mixture. The reaction mixture for forming the reaction product may comprise the nitrosating compound in an amount of from about 1 to about 75 wt. %, alternatively from about 1 to about 10 wt. %, or alternatively from about 10 to about 75 wt. %, based on a total weight of the reaction mixture.

Thus, it should be appreciated that the contemplated nitric oxide precursor is a reaction product of a thiol-containing alcohol, a silane, and a nitrosating compound. In these and other embodiments, the reaction mixture is substantially free of a solvent. In various embodiments, the thiol-containing alcohol and the silane are capable of reacting to form an intermediate, and the intermediate and the nitrosating compound are capable of reacting (typically in the same reaction vessel) to form the nitric oxide precursor.

Most typically, filtration is used to remove the nitrosothiol-containing nitric oxide precursor from the reaction mixture. In some embodiments, the resulting nitric oxide precursor is pulverized or otherwise comminuted to create a fine powder. Alternatively, the nitric oxide precursor may also be used in crystalline form (which may delay or slow down release of nitric oxide).

In various embodiments, the nitric oxide precursor comprises a nitrosothiol that is capable of decomposing to form the nitric oxide. It is contemplated therein that the nitric oxide and air will react, resulting in a mixture containing various oxides of nitrogen. Specifically, the addition of nitric oxide to air, or air to nitric oxide, results in the formation of nitric dioxide when nitric oxide reacts with the oxygen in air. The concentration of each nitrogen-oxide species that is present in a mixture may vary with temperature, pressure, and initial concentration of the nitric oxide.

Nitric oxide is lipid soluble and has the ability to disrupt the lipid membranes of microorganisms, to modulate cell and tissue response, to control coagulation and biological integration, to impart antimicrobial characteristics, etc. Furthermore, nitric oxide may inactivate thioproteins thereby disrupting the functional proteins of microbes. Nitrogen dioxide is more water soluble than nitric oxide. Finally, nitric oxide and nitrogen dioxide are effective disruptors of DNA, causing strand breaks and other damage leading to an inability for the cell to function.

The nitric oxide precursor may be used in a wide variety of medical and consumer applications. The properties of the nitric oxide precursor may be adjusted based on the selection of the thiol-containing alcohol and the silane to suit specific applications. Non-limiting examples of suitable adjustments include nitric oxide generation capabilities and the rate of release of nitric oxide. As described in greater detail below, the nitric oxide precursor may be incorporated into a variety of carriers such that nitric oxide is released from the carrier resulting from decomposition of the nitric oxide precursor. In addition to tuning decomposition of the nitric oxide precursor, release of nitric oxide may also be adjusted based on carrier composition and characteristics to further control release of nitric oxide from the carrier. This controlled nitric oxide release is useful for sterilizing and sanitizing medical and consumer devices.

In some embodiments, the nitric oxide precursor is included in a detergent composition or a cleaning composition. As used herein the phrases “detergent composition” or “cleaning composition” includes compositions and formulations designed for cleaning soiled material. Such compositions include, but are not limited to, object cleaning composition, medical device cleaning compositions, hard surface cleaning compositions, dishware cleaning compositions, laundry cleaning compositions and detergents, spray products, dry cleaning agent or composition, unit dose formulation, delayed delivery formulation, detergent contained on or in a porous substrate or nonwoven sheet, detergent contained on or in a water-soluble film, and other suitable forms that may be apparent to one skilled in the art in view of the teachings herein. The multi-component composition may have a form selected from liquid, powder, single-phase or multi-phase unit dose, 2-layer paper carrier, pouch, tablet, gel, paste, bar, or flake.

The method of forming the nitric oxide precursor can be performed in a single reaction mixture as compared to other conventional methods of forming nitric oxide precursors. The nitric oxide precursor may be under ambient conditions with minimal manipulation of pH. A variety of thiol-containing alcohols (e.g., primary, secondary, or tertiary thiol-containing alcohols) and silanes (e.g., primary, secondary, or tertiary silanes) may be utilized to form the nitric oxide precursor. This variety of reactants allows for the formation of primary, secondary, and tertiary nitrosothiol (RSNOs). The resulting nitric oxide precursor can be easily and quickly coated with a variety of polymers for controlled release of the nitric oxide. The method can incorporate a variety of particle sizes, additives, and carrier/polymers, including blends of the same.

In some embodiments, the contemplated system can take place under ambient conditions with no manipulation of pH. Ambient conditions typically involve an environment with an ambient temperature about 18° C., or about 20° C., or about 22° C. (all +/−3° C.) and an environment with ambient pressure conditions of about 1013.25 millibars (+/−100 mbar). However, it should be recognized that numerous alternate temperatures (higher and lower) are also deemed suitable for use herein.

As mentioned above, the decomposition rate of the nitric oxide precursor will depend on a variety of factors which can be fine-tuned to achieve a desired decomposition profile. Among other factors, the decomposition rate can be modified by selecting the type of resulting nitrosothiol. For example, primary nitrosothiols have a substantially higher rate of decomposition than secondary or tertiary nitrosothiols. In further embodiments, different alcohols containing primary, secondary, or tertiary thiols with variable length chains of alkane, alkyl, or alkyne carbons and heteroatoms can be used to react with a silane to provide differing decomposition rates. Moreover, the primary, secondary, or tertiary thiols can be “R” or “S” configuration.

In various embodiments, the inventive subject matter can also be quickly and easily coupled to or admixed with polymeric material or inorganic material to act as a carrier for the controlled release of NO properties. For example, the nitrosothiol particles or powders can be covered in a thin coating of a synthetic polymeric material such as polyvinyl choride, polyvinyl alcohol, or polydimethylsiloxane to adjust NO-releasing characteristics. Consequently, the bare powders or the coated powders can be blended into a polymer matrix that can then be cast into films or coated onto devices (e.g., to produce a packaging system) to impart controlled nitric oxide releasing properties.

In some embodiments, various packaging systems may comprise a pouch, including but not limited to, Nylon/Nylon pouches, Tyvek®/Mylar® pouches, Foil/Nylon pouches, Foil/Mylar® pouches, and Foil/Foil pouches. In other examples, the package may comprise a thermoformed polyethylene terephthalate-glycol (PETG) tray with a lid. Such lid can be made of synthetic flashspun high-density polyethylene fibers, foil, medical grade paper, or a number of other materials. In further embodiments, the package may be made in whole or in part of flexible material that is woven, stretched, and/or a loose fiber aggregate. As expressed here, “flexible” refers to the ability to be elastically deformable using manual force.

In other embodiments, the synthetic polymeric carrier is configured as a packaging material, for example, a tray, or a film, or a pellet, or a sachet.

In terms of utilizing a sachet, the contemplated composition may be enclosed in a gas permeable sachet and subsequently placed in proximity to a non-sterile object.

In further embodiments the carrier of the nitrosothiol is a natural polymeric material that may be configured as a cloth, or a woven article, or a loose fiber aggregate.

In terms of the polymeric carrier configuration, the polymeric carrier can coat at least 10% of the nitrosothiol compound, or at least 20% of the nitrosothiol compound, or at least 50% of the nitrosothiol compound, or at least 100% of the nitrosothiol compound. Nonetheless, the nitrosothiol compound may be present in the overall composition, including the carrier, in an amount of at least 0.01 wt %, or at least 0.05 wt %, or at least 0.10 wt %, or at least 1.0 wt %, or at least 5.0 wt %, or at least 10 wt %.

As desired, nitrosothiol particles or powders may also be provided in a large variety of sizes and size distribution depending on need, such that they can be mixed or matched in blends to define the NO release profile of the composite material. For example, the compound may be granulated and may have a largest dimension of at least 10 μm, or at least 25 μm, or at least 50 μm, or at least 100 μm, or at least 150 μm, or at least 200 μm, or at least 300 μm, or at least 400 μm, or at least 500 μm.

Additives may optionally be blended into the composite material to add an additional control point for NO release. For example, transition metals, chelators, ascorbic acid, or reducing sugars may be added. In some embodiments, the additive may also be a pH modulator, a hygroscopic agent, a hydrophobic agent, a light filter, or a light sensitizer. Such additives could be present in trace amounts to equimolar ratio, or even be present in molar excess relative to the thiol-containing agent. Preferably, the compound is present in the carrier in an amount sufficient to release antimicrobially effective quantities of NO from the carrier (typically within 1, or 6, or 12, or 24 hours). For example, an antimicrobially effective quantity of NO is one where subsequent sterilization is performed to a sterility assurance level of equal or less than 10⁻⁶.

The inventor further contemplates a method of forming a composite article that is configured to sterilize objects. In some embodiments, the nitrosothiol may be formed or obtained. Most typically, the NO-releasing composition is incorporated into an article that is configured to retain a non-sterile object. Exemplary methods of incorporation involve extruding, or pressing, or combining with a packing material. Consequently, a composite article is formed that is typically configured to allow for delivery of nitric oxide to the article at a concentration sufficient for sterilization.

Various packaging systems may comprise a pouch, including but not limited to, Nylon/Nylon pouches, Tyvek®/Mylar® pouches, Foil/Nylon pouches, Foil/Mylar® pouches, and Foil/Foil pouches. In other examples, the package may comprise a thermoformed polyethylene terephthalate-glycol (PETG) tray with a lid. Such lid can be made of synthetic flashspun high-density polyethylene fibers, foil, medical grade paper, or a number of other materials. In further embodiments, the package may be made in whole or in part of flexible material that is woven, stretched, and/or a loose fiber aggregate. Additional embodiments may include a bag, or a tray, or a flask. As expressed here, “flexible” refers to the ability to be elastically deformable with or without manual force.

The inventor further contemplates a method of sterilization that involves placement of a non-sterile object in a confined space, that most typically further includes the NO-releasing composition discussed above (or that may be made at least in part from the NO-releasing composition discussed above). In some embodiments, the confined space is then sealed such that the non-sterile object and the discussed NO-releasing composition are physically separated from an environment external to the confined space. Preferably, NO is then released into the confined space from the discussed NO-releasing composition.

In various embodiments, the inventor contemplates utilizing the nitric oxide precursor to form nitric oxide for a variety of medical and consumer applications. In various embodiments, an article (e.g., a device or object) may be treated with the nitric oxide precursor, such as in the form of a liquid, a powder, a film, a coating, or the like. Non-limiting examples of suitable uses of the nitric oxide precursor (e.g., as liquids, powders, films, or coatings) include: detergent or cleaning solutions for sanitizing or sterilizing objects treated with the solution (e.g., sports equipment, such as hockey gloves and cycling gloves, cleaning surgical instruments, internal lumens of endoscopes, surfaces of medical equipment, a tissue sample, a biohazardous material, and the like); detergent or cleaning powders for sanitizing or sterilizing objects treated with the powder hygienic containers for sanitizing hygiene devices (e.g., desiccants and the like); medical device containers for sanitizing medical instruments (e.g., stethoscopes, otoscopes, and the like), medical devices (e.g., portable ultrasound device, communication devices, and the like); components of devices exposed to moisture for resisting growth of mold or mildew (e.g., washing machines, boat compartments, and the like); sporting equipment (e.g., yoga mats, touchable surfaces of strength training equipment, touchable surfaces of cardio equipment, and the like); liners for sporting equipment bags for sanitizing sporting equipment (e.g., shoes, hockey equipment, ski equipment, facemasks, googles, helmets, and the like); food packaging for preserving foodstuff (e.g., meats, fruits, vegetables, cheeses, ingredients thereof, and the like); components of vehicles for sanitizing vehicles (e.g., headliners, seat cushion liners, carpet liners, and the like); within drawers of cabinets, desks, boxes, etc., to eliminate musty odors; a kitchen product (e.g., to clean counters, appliances, cooking utensils, and the like); and a cleaning tool (e.g., for use on floors, toilets, sinks, showers, door handles, and the like).

As desired, the confined space may be configured as a bag, or a pouch, or a container with a lid. In some embodiments, the confined space has a volumed of at least 10 cm³, or at least 50 cm³, or at least 100 cm³, or at least 250 cm³, or at least 500 cm³, or at least 750 cm³, or at least 1,000 cm³.

In some embodiments, the NO-releasing composition releases NO in response to a change in temperature, pressure, pH, humidity, illumination with visible or UV light, or a combination thereof.

The inventors contemplate that the nitric oxide precursor may exhibit decomposition to nitric oxide after forming the reaction product within a predetermined amount of time, such as within 1 hour, alternatively within 30 minutes, alternatively within 5 minutes, alternatively within 1 minute, or alternatively within 10 seconds, alternatively within 1 second, or alternatively within 0.1 seconds. Alternatively, the nitric oxide precursor exhibits minimal decomposition to nitric oxide after forming the nitric oxide precursor for a time period of at least 5 minutes, alternatively at least 10 minutes, alternatively at least 15 minutes, alternatively at least 30 minutes, alternatively at least 45 minutes, alternatively at least 1 hour, alternatively at least 2 hours, alternatively at least 3 hours, alternatively at least 4 hours, alternatively at least 5 hours, alternatively at least 6, alternatively at least 7 hours, alternatively at least 8 hours, alternatively at least 9 hours, alternatively at least 10 hours, alternatively at least 11 hours, alternatively at least 12, alternatively at least 13 hours, alternatively at least 14 hours, alternatively at least 15 hours, alternatively at least 16 hours, alternatively at least 17 hours, alternatively at least 18 hours, alternatively at least 19 hours, alternatively at least 20 hours, alternatively at least 21 hours, alternatively at least 22 hours, alternatively at least 23 hours, alternatively at least 24 hours, alternatively at least 48 hours, or alternatively at least 365 days, depending on the specific thiol-containing alcohol and silane used and how the precursor is stored. Alternatively, the nitric oxide precursor may exhibit minimal decomposition to nitric oxide for a time period of from 5 minutes to 365 days, alternatively from 5 minutes to 48 hours, alternatively from 5 minutes to 24 hours alternatively from 1 hours to 24 hours, alternatively from 4 hours to 24 hours, alternatively from 8 hours to 24 hours, alternatively from 16 hours to 24 hours, alternatively from 20 hours to 24 hours after forming the nitric oxide precursor, depending on the specific thiol-containing alcohol and silane used and how the precursor is stored. From a different perspective, the nitric oxide precursor may exhibit decomposition to nitric oxide after forming the reaction product within a predetermined amount of time, such as from about 0.01 seconds to about 1 hour, alternatively from about 0.01 seconds to about 30 minutes, alternatively from about 1 second to about 5 minutes, or alternatively from about 1 second to about 1 minute. In various embodiments, decomposition to nitric oxide may be sustained for a predetermined amount of time, such as a time period of from about 1 minutes to about 1 year, alternatively from about 1 hour to about 6 months, alternatively from about 24 hours to about 3 months, or alternatively from about 1 week to about 8 weeks. From a different perspective, decomposition to nitric oxide may be sustained for a period of at least 1 minute, alternatively at least 1 hours, alternatively at least 24 hours, or alternatively at least 1 week. Without being bound by theory, it is believed that properties of the nitric oxide precursor may be adjusted based on the selection of the thiol-containing alcohol and silane for tuning decomposition to nitric oxide suitable for specific applications.

The nitric oxide precursor may exhibit an improved storage stability as compared to a nitric oxide precursor formed free of the reaction product. In various embodiments, the nitric oxide precursor exhibits minimal degradation at 4° C. for a time period of at least 1 month, alternatively at least 2 months, alternatively at least 3 months, alternatively at least 6 months, or alternatively at least 12 months. In other embodiments, the nitric oxide precursor exhibits minimal degradation at 23° C. for a time period of at least 1 month, alternatively at least 2 months, alternatively at least 3 months, alternatively at least 6 months, or alternatively at least 12 months. The phrase “minimal degradation” means that nitric oxide precursor exhibits minimal color difference over a desired time period of 10% of the ΔE*ab, alternatively 5% of the ΔE*ab, alternatively 4% of the ΔE*ab, alternatively 3% of the ΔE*ab, alternatively 2% of ΔE*ab, alternatively 1% of the ΔE*ab, or alternatively 0.1% of the ΔE*ab in accordance with ASTM D2244-21.

The nitric oxide precursor is capable of decomposing to form nitric oxide. In various embodiments, the nitric oxide precursor comprises a nitrosothiol that is capable of decomposing to form the nitric oxide. It is contemplated herein that nitric oxide is capable of being formed at a temperature of from 0° C. to 100° C., in the presence or absence of visible light, or a combination thereof. To this end, the inventors contemplate that the nitric oxide precursor is capable of decomposing to form the nitric oxide at a predetermined rate and/or for a predetermined amount of time based on the particular combination of the thiol-containing alcohol and the silane utilized along with the temperature of, and amount of light exposure to, the nitric oxide precursor.

With regard to the silane of the reaction product, there is a wide variety of possible silanes that can be used, depending on the design constraints of the desired application of the nitric oxide precursors. Exemplary silanes may have a structure according to the following formulas (I), (II), (III), or combinations thereof:

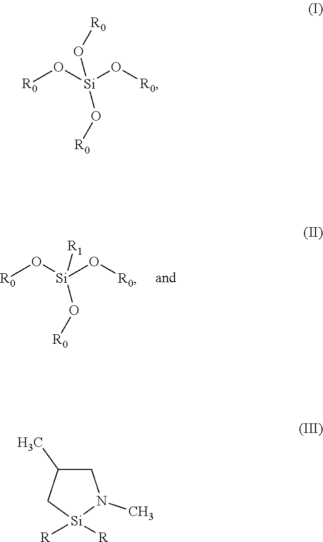

Most typically, each R is, independently, a methyl group, a methylene oxide group, or an ethylene oxide group, each R0 is, independently, a methylene oxide group or an ethylene oxide group, and R1 is a mercapto alkyl group. In certain embodiments, the silane comprises tetramethoxysilane, tetraethoxysilane, or a combination thereof. In an exemplary embodiment, the silane comprises tetraethoxysilane. Therefore, in some embodiments, the silane is a tetraalkoxysilane, or a trialkoxysilane, or a cyclic azasilane.

Other non-limiting examples of suitable silanes include tetraethylorthosilicate (TEOS), a polycondensate of TEOS, methyltrimethoxysilane (MTMS), vinyl-trimethoxysilane, methylvinyldimethoxysilane, dimethyldiethoxysilane, vinyltriethoxysilane, tetra-n-propylorthosilicate, vinyltris(methylethylketoxime)silane, methyltris(methylethylketoxime) silane, trisacetamidomethylsilane, bisacetamidodimethylsilane, tris(N-methyl-acetamido)methylsilane, bis(N-methylacetamido)dimethylsilane, (N-methyl-acetamido)methyldialkoxysilane, trisbenzamidomethylsilane, trispropenoxymethylsilane, alkyldialkoxyamidosilanes, alkylalkoxybisamidosilanes, CH3Si(OC2H5)1-2 (NHCOR) 2-1, (CH3Si(OC2H5) (NCH3COC6H5) 2, CH3Si(OC2H5)-(NHCOC6H5) 2, methyldimethoxy(ethylmethyl-ketoximo)silane; methylmethoxybis-(ethylmethylketoximo)silane; methyldimethoxy(acetal-doximo)silane; methyldimethoxy(N-methylcarbamato)silane; ethyldimethoxy(N-methyl-carbamato)silane; methyldimethoxyisopropenoxysilane; trimethoxyisopropenoxysilane; methyltri-isopropenoxysilane; methyldimethoxy(but-2-ene-2-oxy)silane; methyldimethoxy(1-phenylethenoxy)silane; methyldimethoxy-2 (1-carboethoxypropenoxy)silane; methylmethoxydi-N-methylaminosilane; vinyldimethoxymethylaminosilane; tetra-N,N-diethylaminosilane; methyldimethoxymethylaminosilane; methyltricyclohexylaminosilane; methyldimethoxy-ethylaminosilane; dimethyldi-N,N-dimethylaminosilane; methyldimethoxyisopropylaminosilane dimethyldi-N,N-diethylaminosilane; ethyldimethoxy(N-ethylpropionamido)silane; methyldi-methoxy(N-methylacetamido)silane; methyltris(N-methylacetamido)silane; ethyldimethoxy(N-methylacetamido)silane; methylmethoxybis(N-methylacetamido)silane; methyltris(N-methylbenzamido)silane; trimethoxy(N-methylacetamido)silane; methyldimethoxy(caprolactamo)silane; methyldimethoxyethylacetimidatosilane; methyldimethoxypropylacetimidatosilane; methyldimethoxy(N,N′,N′-trimethylureido)silane; methyldimethoxy(N-allyl-N′,N′-dimethylureido)silane; methyldimethoxy(N-phenyl-N′,N′-dimethylureido)silane; methyldimethoxyisocyanatosilane; dimethoxydiisocyanatosilane; methyldimethoxythioisocyanatosilane; methylmethoxydithioisocyanatosilane, N-(2-aminoethyl)-3-aminopropyltrimethoxysilane, 3-methacryloxypropyltrimethoxysilane, 3-(2-(vinylbenzylamino)ethylamino)-propyltrimethoxysilane, 3-glycidoxypropyltrimethoxysilane, triacetoxyvinylsilane, tris-(2-methoxyethoxy)-vinylsilane, 3-chloropropyltrimethoxysilane, 1-trimethoxysilyl-2-(p,m-chloromethyl)phenylethane, 3-chloropropyltriethoxysilane, N-(aminoethylaminomethyl)phenyltrimethoxysilane, N-(2-aminoethyl)-3-aminopropyl tris(2-ethylhexoxy)silane, 3-aminopropyltrimethoxysilane, trimethoxysilylpropylenetriamine, β(3,4-epoxycyclohexyl)ethyltrimethoxysilane, 3-mercaptopropyltrimethoxysilane, 3-mercaptotriethoxysilane, 3-mercaptopropylmethyldimethoxysilane, bis(2-hydroxyethyl)-3-aminopropyltrimethoxysilane, 1,3-divinyltetramethyldisilazane, vinyltrimethoxysilane, 2-(diphenylphosphino)ethyltriethoxysilane, 2-methacryloxyethyldimethyl[3-trimethoxysilylpropyl]ammonium chloride, 3-isocyanatopropyldimethylethoxysilane, N-(3-acryloxy-2-hydroxypropyl)-3-aminopropyltriethoxysilane, vinyl tris(t-butylperoxy)silane, methyltrimethoxysilane, ethyltrimethoxysilane, phenyltrimethoxysilane, phenyltriacetoxysilane, methyltrimethoxysilane, phenyltrimethoxysilane, or combinations thereof.

Referring back to the thiol-containing alcohol of the reaction product, there is a wide variety of possible thiol-containing alcohols that can be used, depending on the design constraints of the desired application of the nitric oxide precursors. Thiol-containing alcohols may also be referred to in the art as a “mercaptoalcohol” or “hydroxymercaptan.” The thiol-containing alcohol may have a structure according to the following formulas (I), (II), (II), or combinations thereof:

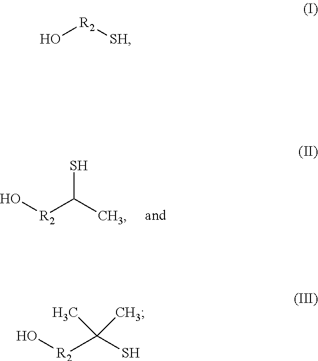

wherein each R₂ is, independently, an alkyl group. In certain embodiments, the thiol-3-containing alcohol comprises 3-mercapto-3-methyl-1-butanol, 2-mercaptoethanol, mercaptohexanol, 4-mercapto-3-methyl-2-butanol, 3-mercapto-2-methyl-1-butanol, 3-mercapto-2-methyl-1-pentanol, (+/−)-4-mercapto-4-methyl-2-pentanol, 2-mercapto-2-methyl-1-pentanol, 4-mercapto-4-methylpentan-2-ol, 3-mercaptohexan-1-ol, mercaptoethanol, 1-mercapto-3-propanol, 1-mercapto-4-butanol, α-mercapto-ω-hydroxyoligoethylene oxides, or combinations thereof. In an exemplary embodiment, the thiol-containing alcohol comprises 3-mercapto-3-methyl-1-butanol.

In certain embodiments, the thiol-containing alcohol includes a thiol-derivatized polymer or filler. It is to be appreciated that the thiol-containing alcohol may be included as part of a peptide or other macromolecules so long as the thiol-containing alcohol is compatible with the components of the reaction mixture of the reaction product.

In various embodiments, when utilized, the thiol-containing alcohol has a weight average molecular weight of no greater than 500,000 g/mol, alternatively no greater than 100,000 g/mol, alternatively no greater than 10,000 g/mol, alternatively no greater than 1,000 g/mol, or alternatively no greater than 500 g/mol. From a different perspective, the thiol-containing alcohol may have a weight average molecular weight of from about 10 g/mol to about 500,000 g/mol, alternatively from about 10 g/mol to about 100,000 g/mol, alternatively from about 10 g/mol to about 1,000 g/mol, or alternatively from about 10 g/mol to about 500 g/mol.

Referring back to the nitrosating compound utilized to form the reaction product, the nitrosating compound may be any compound serving as a source of nitroso groups and will in general be a compound of formula NOX where X is an organic or inorganic anion or a group OR2 where R2 is an organic group. X may thus be an organic anion derived from a carboxylic acid, e.g., an alkane carboxylic acid containing 2-7 carbon atoms, nitrosating agents of this type including acetyl nitrite and propionyl nitrite. Where X is an inorganic anion this may be derived from, for example, a mineral acid, e.g., a halide ion such as chloride or bromide or a sulphate ion, or from a Lewis acid, e.g., a borofluoride ion. Other inorganic anions include hydroxide and sulphonate. Nitrosating compounds of this type thus include nitrosyl chloride, nitrosyl sulphate, nitrosyl borofluoride, nitrous acid and Fremys salt (potassium nitrosyldisulphonate). Where X is a group of formula OR2 the organic group R2 may be, for example, a lower alkyl group, such as containing 1-9 carbon atoms, e.g., ethyl, n-propyl, isopropyl, n-butyl, t-butyl or isopentyl.

In certain embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, nitrite salts, ion paired nitrite, silver nitrite, zinc nitrite, iron nitrite, copper nitrite, transition metal-nitrite compounds, or combinations thereof. Also, nitric oxide gas may be used as a nitrosating agent.

In various embodiments, the nitrosating compound has a weight average molecular weight of no greater than 10,000 g/mol, alternatively no greater than 1,000 g/mol, alternatively no greater than 500 g/mol, or alternatively no greater than 250 g/mol. From a different perspective, the nitrosating compound may have a weight average molecular weight of from about 10 g/mol to about 10,000 g/mol, alternatively from about 10 g/mol to about 1,000 g/mol, alternatively from about 10 g/mol to about 500 g/mol, or alternatively from about 10 g/mol to about 250 g/mol. For example, NaNO2 has a weight average molecular weight of 69 g/mol and butyl nitrite has a weight average molecular weight of 103 g/mol. Without being bound by theory, it is believed that reaction kinetics of forming the reaction product is improved by utilizing the nitrosating compounds having lower weight average molecular weights.

In various embodiments, the thiol-containing alcohol and the silane may be reacted in the presence of an acid. If utilized, the catalyst may be included in various amounts. The acid may be utilized to improve formation and/or stability of the reaction product, such as when utilizing primary amine comprising amino acids, such as cysteine. In certain embodiments, the acid may comprise hydrochloric acid. Other non-limiting examples of suitable acids includes citric acid, methane sulfonic acid, ethane sulfonic acid, propane sulfonic acid, butane sulfonic acid, acetic acid, hydroxy acetic acid, propionic acid, hydroxy propionic acid, a-ketopropionic acid, butyric acid, mandelic acid, valeric acid, succinic acid, tartaric acid, malic acid, oxalic acid, fumaric acid, adipic acid, maleic acid, sorbic acid, benzoic acid, succinic acid, glutaric acid, adipic acid, α-hydroxy acids, ethylenediaminetetraacctic acid (EDTA), phosphonic acid, octyl phosphoric acid, acrylic acid, polyacrylic acid, aspartic acid, polyaspartic acid, p-hydroxybenzoic acids, iminoacetic acids, or combinations thereof. It is to be appreciated that the acid may be included as part of any component of the composition (e.g., carrier, solvent, etc.) or the reactants of the reaction product.

In certain embodiments, the thiol-containing alcohol and the silane are reacted in the presence of a catalyst. If utilized, the catalyst may be included in various amounts. The catalyst may comprise any suitable catalyst or mixtures of catalysts known in the art. In certain embodiments, the catalyst may comprise a transition metal catalyst (e.g., an organotin catalyst). In exemplary embodiments, the catalyst comprises a dimethyldineodecaneoate tin catalyst.

Other non-limiting examples of suitable catalysts include metal catalysts, amine catalysts, and a combination thereof. Examples of suitable metal catalysts include tin, iron, lead, bismuth, mercury, titanium, hafnium, zirconium, iron (II) chloride, zinc chloride, lead octoate stabilized stannous octoate, tin (II) salts of organic carboxylic acids such as tin (II) acetate, tin (II) octoate, tin (II) ethylhexanoate and tin (II) laurate, and dialkyltin (IV) salts of organic carboxylic acids such as dibutyltin dilaurate, dibutyltin diacetate, dibutyltin maleate and dioctyltin diacetate, and combinations thereof. In certain embodiments, the polymerization catalyst component comprises dimethylethanolamine. Examples of suitable amine catalysts include amidines such as 2,3-dimethyl-3,4,5,6-tetrahydropyrimidine, tertiary amines such as triethylamine, tributylamine, dimethylbenzylamine, N-methylmorpholine, S-ethylmorpholine, N-cyclohexylmorpholine, N,N,N′,N′-tetramethylethylenediamine, N,N,N′,N′-tetramethylbutanediamine, N,N,N′,N′-tetamethylhexane-1,6-diamine, pentamethyldiethylenetriamine, bis(dimethylaminoethyl) ether, bis(dimethylaminopropyl) urea dimethylpiperazine, 1,2-dimethylimidazole, 1-azabicyclo[3.3.0]octane and typically 1,4-diazabicyclo[2.2.2]octane, and alkanolamine compounds such as triethanolamine, triisopropanolamine, N-methyldiethanolamine and N-ethyldiethanolamine, dimethylethanolamine, and combinations thereof.

In various embodiments, the reaction mixture is substantially free of a solvent. Alternatively, the reaction product is formed substantially free of the presence of a solvent. The phrase “substantially free” as used herein refers to either the complete absence of the acid or a minimal amount thereof merely as impurity, unintended byproduct of another ingredient, or in an amount that has a negligible impact on the composition or the nitric oxide precursor. In certain embodiments, “substantially free” means that the solvent is present in the reaction mixture in an amount of less than 0.5 wt. %, less than 0.25 wt. %, less than 0.1 wt. %, less than 0.05 wt. %, or less than 0.01 wt. %, or even 0 wt. %, based on a total weight of the reaction mixture.

In other embodiments, the thiol-containing alcohol and the silane may be formed in the presence of a solvent. If utilized, the solvent may be included in various amounts. In certain embodiments, the solvent may comprise an organic solvent, such as tetrahydrofuran. Other non-limiting examples of suitable solvents include aromatics, aliphatics, ketones, such as methyl ethyl ketone, isobutyl ketone, ethyl amyl ketone, acetone, alcohols, such as methanol, ethanol n-butanol isopropanol esters, such as ethyl acetate, glycols, such as ethylene glycol propylene glycol ethers, such as tetrahydrofuran, ethylene glycol mono butyl ether, or combinations thereof.

A method of sterilizing or sanitizing an article (e.g., a device or object) is also provided herein. The method comprises applying the nitric oxide precursor described above to the article. A method of forming the nitric oxide precursor is also provided. The method comprises combining a thiol-containing alcohol, a silane, and a nitrosating compound to form a reaction mixture, and allowing the reaction mixture to react for a time period of at least 1 hour, alternatively at least 4 hours, alternatively at least 8 hours, or alternatively at least 12 hours. In some embodiments, the step of combining is further defined as combining the thiol-containing alcohol, the silane, the nitrosating compound, the acid, and the catalyst to form the reaction mixture.

Viewed from a different perspective, the nitric oxide precursor may be incorporated in or with a carrier to form a composite article (e.g. a film, a coating, or a coated particle). The composite article is capable of providing nitric oxide via decomposition of the nitric oxide precursor to form nitric oxide. To this end, the inventors contemplate that the nitric oxide precursor of the composite article is capable of decomposing to form the nitric oxide at a predetermined rate and/or for a predetermined amount of time based on the particular combination of the thiol-containing alcohol and the silane utilized, the temperature of, and amount of light exposure to, the nitric oxide precursor, and the chemical and physical configurations of the carrier, mechanical properties of the carrier, surface chemistry of the carrier, hydrophobicity, and the like.

In certain embodiments, the composite article may exhibit minimal release of nitric oxide for a time period of at least 5 minutes hours after forming the nitric oxide precursor. In various embodiments, the composite article exhibits minimal release of nitric oxide after forming the nitric oxide precursor for a time period of at least at least 10 minutes, alternatively at least 15 minutes, alternatively at least 30 minutes, alternatively at least 45 minutes, alternatively at least 1 hour, alternatively at least 2 hours, alternatively at least 3 hours, alternatively at least 4 hours, alternatively at least 5 hours, alternatively at least 6, alternatively at least 7 hours, alternatively at least 8 hours, alternatively at least 9 hours, alternatively at least 10 hours, alternatively at least 11 hours, alternatively at least 12, alternatively at least 13 hours, alternatively at least 14 hours, alternatively at least 15 hours, alternatively at least 16 hours, alternatively at least 17 hours, alternatively at least 18 hours, alternatively at least 19 hours, alternatively at least 20 hours, alternatively at least 21 hours, alternatively at least 22 hours, alternatively at least 23 hours, alternatively at least 24 hours, alternatively at least 48 hours, or alternatively at least 365 days, depending on the specific thiol-containing alcohol and silane used and how the precursor is stored.

The carrier material may be thermoplastic or thermoset. Non-limiting examples of suitable thermoplastic materials include polyvinyl chloride (“PVC”), polyethylene terephthalate (“PET”), polyethylene terephthalate glycol-modified (“PETG”), polypropylene (“PP”), polyethylene (“PE”), polyamide, such as nylon, and combinations thereof. Non-limiting examples of suitable thermoset materials include UV curable materials, heat curable materials, chemical curable materials, such as free radical, room temperature curable materials, and cold cured materials. In some embodiments, the carrier comprises an aqueous-based heat-sealable adhesive coating that may be formed from a polymeric material, such as ethylene vinyl acetate, ethylene-acrylate copolymers, and polyurethanes. The aqueous-based heat-sealable adhesive coating may comprise basic, neutral, or acidic pH solutions. Non-limiting examples of suitable aqueous-based heat-sealable adhesive coatings are disclosed in WO 2015/160939A1, which is incorporated by reference in its entirety.

In certain embodiments, the carrier is formed from a cellulose, a polyvinyl chloride, a polyurethane, a carbosil, a polydimethylsiloxane, a phenyl carboxyl polydimethylsiloxane, an acrylic polymer, a polyester, a poly (lactic acid), a poly (lactic-co-glycolic acid), poly(vinyl acetate), ethylene vinyl acetate, tecothane, pellethane, a hydrogel, a polytetrafluoroethylene, a copolymer thereof, or combinations thereof. In one exemplary embodiment, the carrier is formed from a polyvinylchloride. Such carriers are well known to those of skill in the art and are described in textbooks such as Remington's Pharmaceutical Sciences, Mack Publishing Co., Easton, Pa., 1985, which is incorporated by reference in its entirety.

In another exemplary embodiment, the carrier is formed from a hydrogel selected from the group of a polymacron, a polyacrylamide, a collagen, an agarose, a hyaluronic acid, a poly (organophosphazenes), a chitosan, a poly(ethylene glycol), poly(vinyl alcohol), and combinations thereof. Non-limiting examples of suitable hydrogels are described in a journal article titled “S-Nitrosothiol Detection via Amperometric Nitric Oxide Sensor with Surface Modified Hydrogel Layer Containing Immobilized Organoselenium Catalyst” cited as Langmuir 2006, 22, 25, 10830-10836, which is incorporated by reference in its entirety.

In further contemplated aspects, a composite article may therefore comprise the nitric oxide precursor in an amount of from about 5 to about 25 wt. %, alternatively from about 20 to about 60 wt. %, or alternatively from about 40 to about 90 wt. %, based on a total weight of the composite article. The composite article may comprise the carrier in an amount of from about 10 to about 95 wt. %, alternatively from about 10 to about 80 wt. %, or alternatively from about 80 to about 95 wt. %, based on a total weight of the composite article.

In further embodiments, the carrier may have a permeability in an amount of at least 0.001, alternatively at least 0.01, alternatively at least 0.1, alternatively at least 1, or alternatively at least 5 g/(m·s·Pa) in accordance with ASTM E2945-14 (2021) for permitting movement of the nitric oxide.

The inventors contemplate that the composite article may further comprise a variety of additives, including, but not limited to, ascorbate, reducing equivalents, oxidizing equivalents, acids, bases, pH buffers, ionophores, enzymes, any agent that will impact the formation and stability of thiols (e.g., including disulfide formation or breaking of disulfide bonds), nitrosothiols (e.g., acids/bases, ion mobility, gas permeability, reaction/buffering of NO gas), plasticizers, surfactants, colorants, fillers, or combinations thereof.

When utilized, the plasticizer may include a plasticizer that is used to modify various characteristics including, but not limited to, permeability, modifying hydrophobicity, tensile strength, elongation, and the like. The plasticizer includes, but is not limited to, phthalates, trimellitates, benzoates, adipates, sebacates, maleates, citrates, epoxidized vegetable oils, sulfonamides, organophosphates, glycols/polyethers, polymeric plasticizers and polybutenes, or combinations thereof. However, it is to be appreciated that the plasticizer may include any other plasticizer understood in the art so long as the plasticizer is compatible with the components of the functionalized polymeric material.

The plasticizer may be an ester plasticizer. Examples of suitable ester plasticizers include, but are not limited to, dioctyl phthalate (DOP), n-hexyl-n-decyl phthalate (NHDP), n-octyl-decyl phthalate (NODP), di(isononyl)phthalate (DINP), di(isodecyl)phthalate (DIDP), diundecyl phthalate (DUP), di(isotridecyl)phthalate (DTDP), di-2-ethylhexyl adipate (DOA), di-n-octyl-n-decyl adipate (DNODA), diisononyl adipate (DINA), di-2-ethylhexyl azelate (DOZ), di-2-ethylhexyl sebacate (DOS), trioctyl trimellitate (TOTM), trioctyl phosphate (TOP), tricresyl phosphate (TCP), aliphatic polyester plasticizer, aliphatic polyol plasticizer, or combinations thereof. In certain embodiments, the plasticizer component includes trioctyl trimellitate (TOTM). It is to be appreciated the plasticizer may include any phthalate known in the art so long as it is compatible with the composite article.

When utilized, the surfactant may include anionic surfactants, non-ionic surfactants, cationic surfactants, Zwitterionic surfactants, or combinations thereof. However, it is to be appreciated that the surfactant may include any other surfactant understood in the art so long as the surfactant is compatible with the components of the composite article.

Examples of suitable anionic surfactants include, but are not limited to, fatty alcohol sulphates, alkylphenol sulphates, fatty alcohol ether sulphates, fatty alcohol ether sulphates, alkylphenol ether sulphates, alkylbenzene sulphonic acid, alkyl ether carboxylic acid and salts thereof, alkyl sulphosuccinates, alkyl sulphosuccinamates, phosphate esters, α-olefin sulphonates, or combinations thereof. Examples of suitable non-ionic surfactants include, but are not limited to, alcohol ethoxylates, alkylphenol ethoxylates, polyethylene oxide/polyethylene oxide block copolymers, polyvinyl alcohol, polyvinyl pyrroliddone, sorbitan fatty acid esters, sorbitan ester ethoxylates, or combinations thereof. Examples of suitable cationic surfactants includes, but are not limited to, alkyl dimethylamines, quaternary ammonium compounds, or combinations thereof. In certain embodiments, the surfactant component includes a nonionic surfactant. The nonionic surfactant may include an acetylene glycol surfactant, 2-ethylhexanol, or a combination thereof.

When utilized, the filler may include any filler that may be used for various objectives including, but not limited to, cost control, rheology control, lubricity modification, as well as to prevent seizing or galling. The filler component may include an inorganic filler. Examples of suitable inorganic fillers include, but are not limited to, powdered nickel, copper, zinc, and aluminum. Suitable mineral fillers include, but are not limited to, talc, calcium carbonate, silicates such as mica, wollastonite, titanium dioxide, quarts, fumed silica precipitated silica, graphite, boron nitride, or combinations thereof. Also included are modifiers such as stearates including zinc stearate, magnesium stearate, sodium stearate, etc.

Other components that may be present in the composite article include minor amounts of antioxidants, inhibitors, defoamers, dispersing aids, heat stabilizers, UV stabilizers, and the like, such as one or more components described in U.S. Patent App. Pub. No. 2004/0258922 A1, in U.S. Pat. No. 9,404,015 B2, and U.S. Pat. No. 10,214,668 B2, the disclosures of which are incorporated herein by reference in their entirety. In various embodiments, one or more of such additives are individually present in the composite article in an amount less than about 5 wt. % based on a total weight of the composite article.

A method of forming the composite article is also provided. The composite article can be formed utilizing conventional techniques understood in the art. The method comprises providing the carrier (e.g., polyvinyl chloride), providing the nitric oxide precursor, and combining the carrier and the nitric oxide precursor to form the composite article. In various embodiments, the method further comprises providing the solvent (e.g., tetrahydrofuran) and the plasticizer (e.g., diisononyl phthalate). In these embodiments, the step of combining comprises combining the solvent, the carrier, and the plasticizer to form a mixture, and combining the mixture and the nitric oxide precursor to form the composite article. The method may further comprise removing excess mixture from the nitric oxide precursor using vacuum filtration to form the composite article. Furthermore, the method may comprise drying the composite article. The composite article may be air dried for a time period of from 1 minutes to 24 hours, or even more. The composite article may be stored in an airtight container at room temperature or 2-8° C.

In these and other embodiments, the detergent or cleaning composition further comprises a cleaning agent. The cleaning agent comprises a detergent, an enzyme, or a combination thereof. A non-limiting example of a suitable cleaning agent include Alconox powder. In embodiments when the cleaning agent comprises a detergent, the detergent may comprise a surfactant, such as amine oxide, alkylbenzene sulfonate, alkyl ether sulfate, fatty alcohol ethoxylate, alkyl glycosides, alkoxylated fatty acid alkyl esters, amine oxides, fatty acid alkanolamides, hydroxy mixed ethers, sorbitan fatty acid esters, polyhydroxy fatty acid amides, and alkoxylated alcohols.

In embodiments when the cleaning agent comprises an enzyme, the enzyme may comprise one or more enzymes, which can display a catalytic activity in a detergent, such as a protease, amylase, lipase, cellulase, hemicellulase, mannanase, pectin-cleaving enzyme, tannase, xylanase, xanthanase, β-glucosidase, carrageenase, perhydrolase, oxidase, oxidoreductase, and mixtures thereof. In certain embodiments, the enzyme comprises proteases, amylases (e.g., α-amylases), cellulases, lipases, hemicellulases, pectinases, mannanases, β-glucanases, or combinations thereof. The properties of the enzyme should be compatible with the multi-component composition (i.e. pH-optimum, compatibility with other enzymatic and non-enzymatic ingredients, etc.). If utilized, the enzyme should be present in effective amounts.

When utilized, the protease may be of animal, vegetable or microbial origin, including chemically or genetically modified mutants. Microbial origin is preferred. It may be an alkaline protease, such as a serine protease or a metalloprotease. A serine protease may for example be of the Si family, such as trypsin, or the S8 family such as subtilisin. A metalloproteases protease may for example be a thermolysin from e.g., family M4, M5, M7 or M8.

When utilized, suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g. H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131:253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO2007/087508 and WO 2009/109500, which are incorporated by reference in their entirety.

When utilized, suitable amylases include those of bacterial or fungal origin. Chemically modified or protein engineered mutants are included. Amylases include, for example, α-amylases obtained from Bacillus, e.g., a special strain of Bacillus licheniformis, described in more detail in GB 1,296,839. Examples of useful amylases are the variants described in WO 94/02597, WO 94/18314, WO 96/23873, and WO 97/43424, which are incorporated by reference in their entirety.

When utilized, suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757, and WO 89/09259, which are incorporated by reference in their entirety.

When utilized, suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257, which are incorporated by reference in their entirety.

The detergent or cleaning composition may further include additives, such as builders, bleaching agents, electrolytes, nonaqueous solvents, pH adjusting agents, fragrances, perfume carriers, fluorescent agents, dyes, hydrotropes, foam inhibitors, silicone oils, antiredeposition agents, graying inhibitors, shrinkage preventers, anti-creasing agents, dye transfer inhibitors, antimicrobial substances, germicides, fungicides, antioxidants, preservatives, corrosion inhibitors, antistatic agents, bitter agents, ironing aids, hydrophobizing and impregnating agents, swelling and anti-slip agents, softening components, and UV absorbers.

The detergent or cleaning composition may comprise the nitric oxide precursor in an amount of from about 0.01 to about 40 wt. %, alternatively from about 0.01 to about 5 wt. %, alternatively from about 5 to about 10 wt. %, or alternatively from about 10 to about 40 wt. %, based on a total weight of the composition. The detergent composition or a cleaning composition may comprise the cleaning agent in an amount of from about 1 to about 99 wt. % based on a total weight of the composition. The detergent composition or a cleaning composition may comprise the solvent in an amount of from about 1 to about 99 wt. % based on a total weight of the composition.

In various embodiments, the detergent or cleaning composition described herein can be filled into a water-soluble envelope and thus be part of a water-soluble package. The water-soluble envelope may be formed by a water-soluble film material. Such water-soluble packages can be produced either by vertical form fill seal (VFFS) methods or by thermoforming methods. In certain embodiments, the nitric oxide precursor may be incorporated into the water-soluble film material, may be disposed in the envelope formed by the water-soluble film material, or both.

The envelope can be made of one layer or of two or more layers of the water-soluble film material. The water-soluble film material of the first layer and of the other layers, if present, can be the same or different. The water-soluble envelope, for example, is made from a water-soluble film material selected from the group comprising polymers or polymer mixtures. The water-soluble envelope may contain polyvinyl alcohol or a polyvinyl alcohol copolymer. Polymers selected from the group comprising acrylic acid-containing polymers, polyacrylamides, oxazoline polymers, polystyrene sulfonates, polyurethanes, polyesters, polyether polylactic acid, and/or mixtures of the above polymers, can be added to a film material that is suitable for producing the water-soluble envelope.

The thermoforming method generally includes forming a first layer from a water-soluble film material to create convexities for receiving a composition therein, filling the composition into the convexities, covering the convexities filled with the composition with a second layer of a water-soluble film material, and sealing the first and second layers together at least around the convexities. The water-soluble package comprising the liquid washing and the water-soluble envelope can have one or more chambers. The water-soluble packages can have a substantially dimensionally stable spherical and pillow-shaped configuration with a circular, elliptical, square, or rectangular basic form. The chambers may be isolated from one another.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1: Nitric Oxide-Releasing Particles

The following components were mixed in one vial to form a reaction mixture: 2 mL of a silane (tetraethoxysilane), 0.5 mL of a thiol-containing alcohol (3-mercapto-3-methyl-1-butanol), 50 μl of an acid (10% acetic acid), 10 μl of a catalyst (20 mg/mL di-n-butyldilauryltin), and 0.75 mL of a nitrosating compound (cleaned tert-butyl nitrite).

The reaction mixture was fully mixed and then left at room temperature open to air in a fume hood and allowed to react overnight. After the reaction, a nitric oxide precursor was formed as a crystal-like product. The crystal-like product was ground to powder with a mortar and pestle to form a nitric oxide precursor powder, which was stored in an air-tight vial at room temperature or 2-8° C.

### Example 2: Polyvinylchloride (PVC) Coated Nitric Oxide-Releasing Particles

A carrier (polyvinylchloride) was dissolved in solvent (tetrahydrofuran) to a final concentration of 0.5% (w/v) with plasticizer (diisononyl phthalate) to a final mass ratio of 3:1 for the carrier to the plasticizer) to form a PVC solution. Nitric oxide-releasing particles obtained from Example 1 were placed on a filter paper. The particles were washed with the 0.5% PVC solution while under vacuum filtration to quickly remove excess PVC solution to form PVC coated nitric oxide-releasing particles. The PVC coated particles were allowed to air dry. The PVC coated nitric oxide-releasing particles were stored in an air-tight vial at room temperature or 2-8° C.

### Example 3: Exemplary and Comparative Particles and Composite Articles

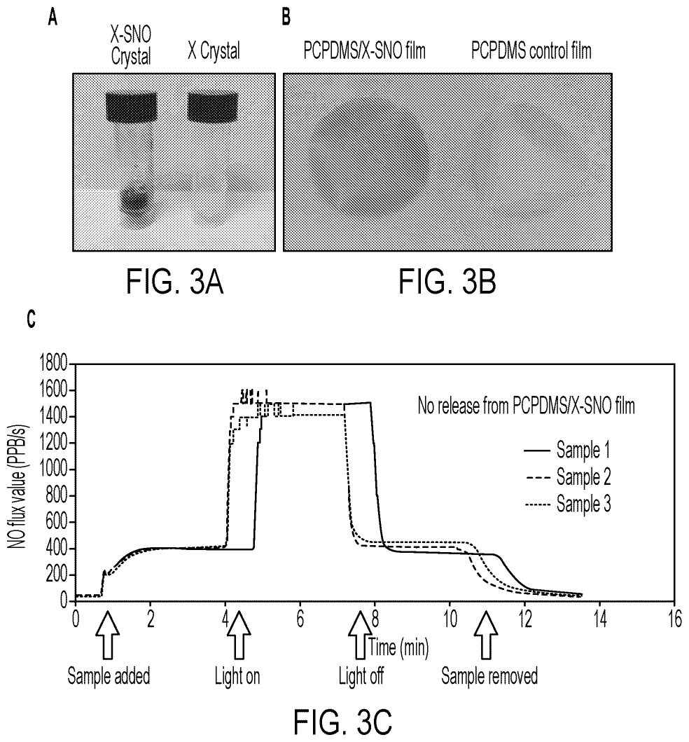

FIG. 3 provides photographs of (A) Nitric oxide releasing silane compared to same material without RSNO, (B) PCPDMS film containing the ground compound in powder form compared to the control powder, shown in A, and (C) nitric oxide release from the film containing the NO releasing powder using light to trigger NO generation.

### Example 4: Exemplary and Comparative Particles and Composite Articles

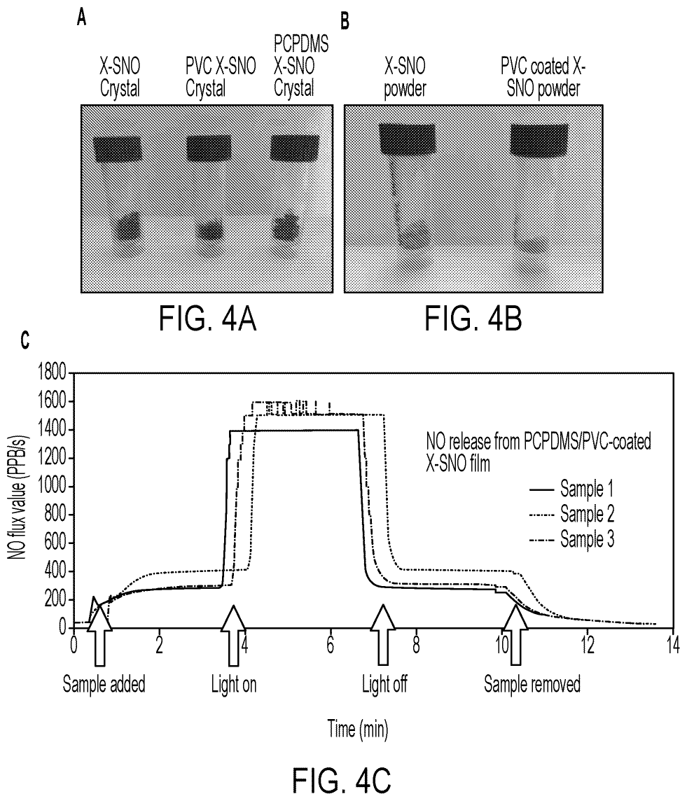

FIG. 4 provides photographs of (A) Nitric oxide releasing silane compounds uncoated, coated in PVC, and coated in PCPDMS, (B) uncoated powder and powder coated in PVC, and (C) nitric oxide release from the film containing the NO releasing uncoated and coated powder using light to trigger NO generation.

### Example 5: Exemplary and Comparative Particles and Composite Articles

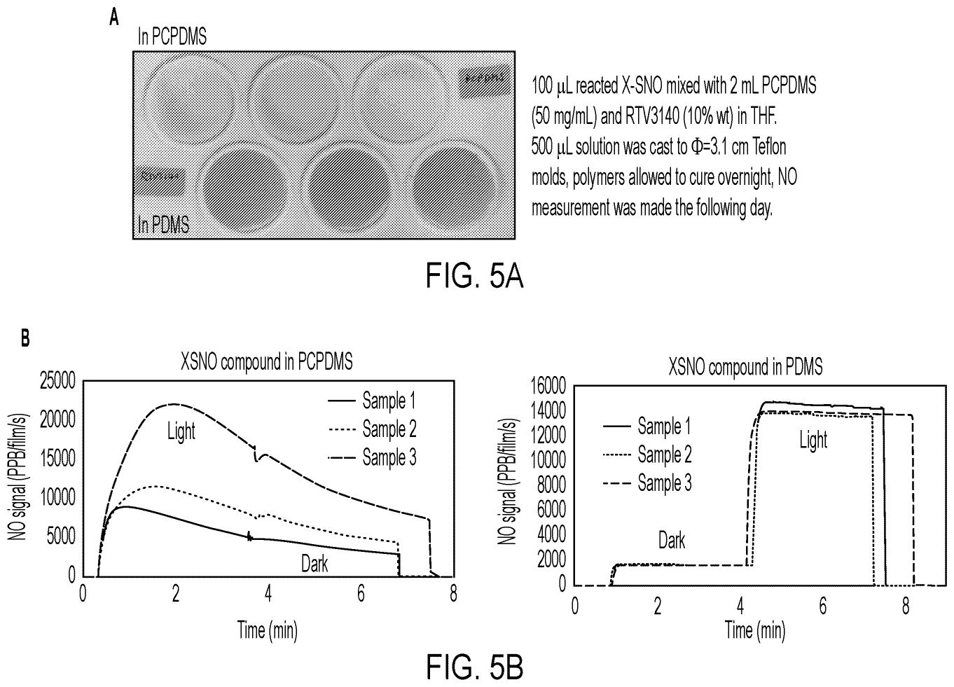

FIG. 5 provides photographs of (A) Nitric oxide releasing silane compounds in PCPDMS and PDMS, and (B) nitric oxide release from the films containing the NO releasing compounds using light to trigger NO generation. The silane compounds were mixed with the polymer solutions prior to compete crosslinking (and crystallization).

### Example 6: Exemplary and Comparative Particles and Composite Articles

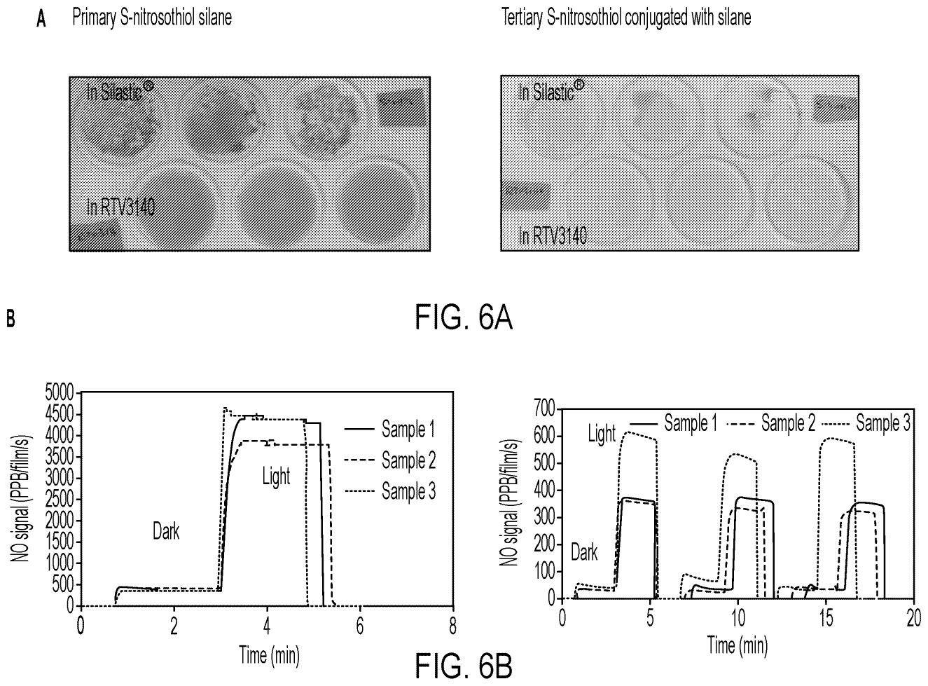

FIG. 6 provides photographs of (A) Nitric oxide releasing silane compounds (primary (red) and tertiary (green)) in Silastic adhesive and RTV-3140 (PDMS), and (B) nitric oxide release from the films containing the NO releasing compounds using light to trigger NO generation. The silane compounds were mixed with the polymer solutions prior to complete crosslinking (and crystallization).

In some embodiments, the numbers expressing quantities of ingredients, properties such as concentration, reaction conditions, and so forth, used to describe and claim certain embodiments of the invention are to be understood as being modified in some instances by the term “about.” As used herein, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed. The recitation of ranges of values herein is merely intended to serve as a shorthand method of referring individually to each separate value falling within the range. Unless otherwise indicated herein, each individual value is incorporated into the specification as if it were individually recited herein.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

1. A composition comprising:
a carrier comprising a polymeric material or an inorganic material coupled to or in admixture with a compound having a structure according to Formula I; 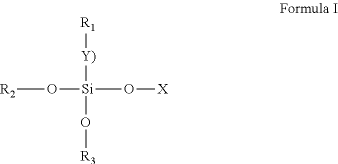
wherein X is selected from the group consisting of 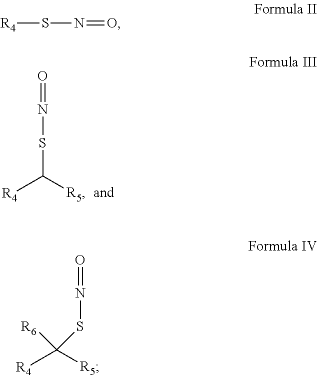
wherein X is covalently bound to O in Formula I via the R4 group in Formula II-IV;
wherein Y is O or null;
wherein each of R₁, R₂, R₃, R₄, R₅, and R₆ are independently selected from the group consisting of hydrogen, alkyl, alkoxy, alkenyl, alkynyl, cycloalkyl, hetercycloalkyl, and aryl, and optionally wherein each of R₁, R₂, R₃, R₄, R₅, and R₆ are, when not a hydrogen, independently substituted with a thiol group, an epoxy group, a nitro group, and/or an amino group.

2. The composition of claim 1, wherein the carrier is a polymeric carrier that at least partially coats the compound, optionally wherein the carrier is a synthetic polymeric carrier that is configured as a packaging material, a film, or a pellet, or optionally wherein the carrier is a natural polymeric carrier that is configured as a fiber, a cloth, or a woven article.

3. (canceled)

4. (canceled)

5. The composition of claim 1, wherein the compound is granulated and has a largest dimension of no more than 500 μm, and/or wherein the compound is present in the composition in an amount of between 0.01 wt % and 0.1 wt %.

6. (canceled)

7. The composition of claim 1, wherein the compound is present in the carrier in an amount sufficient to release antimicrobially effective quantities of nitric oxide from the carrier.

8. The composition of claim 1, wherein X has a structure according to Formula II.

9. The composition of claim 1, wherein X has a structure according to Formula III.

10. The composition of claim 1, wherein X has a structure according to Formula IV.

11. The composition of claim 1, wherein the composition comprises a compound of Formula I in which X has a structure according to one of Formula II, III, or IV, and further comprises an additional compound of Formula I in which X has a structure according to one of Formula II, III, or IV, wherein X in the compound and X in the additional compound are not the same.

12. The composition of claim 1, wherein the composition comprises a compound of Formula I in which Y is null, and further comprises an additional compound of Formula I in which Y is O.

13. The composition of claim 11, wherein Y in the compound of Formula I is null, and wherein Y in the additional compound is O.

14. (canceled)

15. (canceled)

16. (canceled)

17. A method of forming a nitrosothiol-containing nitric oxide precursor, comprising:
combining in a reaction vessel a thiol-containing alcohol, a silane, and a nitrosating compound to form a reaction mixture; and
reacting in the reaction mixture the thiol-containing alcohol with the silane to thereby form a thiol-containing intermediate and reacting in the reaction mixture the thiol-containing intermediate with the nitrosating compound to thereby form the nitrosothiol-containing nitric oxide precursor.

18. (canceled)

19. (canceled)

20. (canceled)

21. The method of claim 17, wherein the thiol-containing alcohol is selected form the group consisting of 3-mercapto-3-methyl-1-butanol, 2-mercaptoethanol, 3-mercaptohexanol, 4-mercapto-3-methyl-2-butanol, 3-mercapto-2-methyl-1-butanol, 3-mercapto-2-methyl-1-pentanol, (+/−)-4-mercapto-4-methyl-2-pentanol, 2-mercapto-2-methyl-1-pentanol, 4-mercapto-4-methylpentan-2-ol, 3-mercaptohexan-1-ol, mercaptoethanol, 1-mercapto-3-propanol, 1-mercapto-4-butanol, and an α-mercapto-ω-hydroxyoligoethylene oxide.

22. The method of claim 17, wherein the silane is a tetraalkoxysilane, a trialkoxysilane, or a cyclic azasilane, or wherein the silane is selected from the group consisting of tetraethylorthosilicate (TEOS), methyltrimethoxysilane (MTMS), vinyl-trimethoxysilane, methylvinyldimethoxysilane, dimethyldiethoxysilane, vinyltriethoxysilane, tetra-n-propyl-orthosilicate, trisacetamidomethylsilane, bisacetamidodimethylsilane, methylmethoxybis-(ethylmethylketoximo)silane, methyldimethoxy-ethylaminosilane; dimethyldi-N,N-di-methylaminosilane, methyldimethoxyisopropylaminosilane triacetoxyvinylsilane, tris-(2-methoxyethoxy)-vinylsilane, 3-chloropropyltriethoxysilane, 3-mercaptotriethoxysilane, ethyltrimethoxysilane, phenyltriacetoxysilane, methyltrimethoxysilane, and phenyltri-methoxysilane.

23. (canceled)

24. The method of claim 17, wherein the nitrosating compound is an inorganic nitrite salt, or wherein the nitrosating compound is selected from the group consisting of sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, an ion paired nitrite, silver nitrite, zinc nitrite, iron nitrite, copper nitrite, and a transition metal-nitrite compound.

25. (canceled)

26. The method of claim 17, wherein the steps of reacting the thiol-containing alcohol with the silane and reacting the thiol-containing intermediate with the nitrosating compound does not require a solvent to form the nitrosothiol-containing nitric oxide precursor, and/or wherein the steps of reacting the thiol-containing alcohol with the silane and reacting the thiol-containing intermediate with the nitrosating compound are performed without adjustment of pH, and/or wherein the steps of reacting the thiol-containing alcohol with the silane and reacting the thiol-containing intermediate with the nitrosating compound are performed at ambient temperature and ambient pressure.

27. (canceled)

28. (canceled)

29. (canceled)

30. The method of claim 17, further comprising adding at least one of an acid and a catalyst to the reaction mixture.

31. (canceled)

32. The method of claim 17, wherein the nitrosothiol-containing nitric oxide precursor has a structure according to Formula I of claim 1.

33. A method of sterilization, comprising:
placing a non-sterile object in a confined space, wherein the confined space further includes the NO-releasing composition of claim 1;
sealing the confined space, wherein the non-sterile object and the NO-releasing composition of claim 1 are physically separated from an environment external to the confined space; and
releasing nitric oxide (NO) into the confined space from the NO-releasing composition of claim 1.

34. (canceled)

35. (canceled)

36. (canceled)

37. (canceled)

38. (canceled)

39. The method of claim 33, wherein the NO-releasing composition of claim 1 releases NO in response to a change in temperature, pressure, pH, humidity, illumination with visible or UV light, or a combination thereof.

40. (canceled)

41. (canceled)

42. The method of claim 33, wherein sterilization is performed to a sterility assurance level of equal or less than 10⁻⁶.

43-50. (canceled)
